(19) United States
(12) Patent Application Publication
(10) Pub. No.: US 2012/0143244 A1
(43) Pub. Date: Jun. 7, 2012

(54) CLOSURE DEVICE

(75) Inventors: Jason P. Hill, Brooklyn Park, MN (US); Mark L. Jenson, Greenfield, MN (US); Michael Whetstone, Co. Durham (GB); Alison Liddy, Galway (IE)

(73) Assignee: Boston Scientific Scimed, Inc., Maple Grove, MN (US)

(21) Appl. No.: 13/310,495

(22) Filed: Dec. 2, 2011

Related U.S. Application Data
(60) Provisional application No. 61/419,570, filed on Dec. 3, 2010.

(51) Int. Cl. A61B 17/08; B23P 11/00; B23P 17/04; A61B 17/10

(52) U.S. Cl. 606/213; 29/428; 29/458

(57) ABSTRACT

The present disclosure provides closure devices for closing an opening in a body vessel, systems for closing an opening in a body vessel, and methods of making a closure device for closing an opening in a body vessel. The closure device may include a plug, an intravascular anchor, a suture that couples the plug to the intravascular anchor and a cinching element that provides a compressive axial force to the plug, wherein the plug and cinching element are positioned over a length of the suture and the suture is secured to the intravascular anchor with a fastening portion embedded in the intravascular anchor.

### PRIORITY INFORMATION

This application claims priority to U.S. Provisional Application No. 61/419,570 filed on Dec. 3, 2010, the specification of which is incorporated herein by reference.

### FIELD OF DISCLOSURE

Embodiments of the present disclosure are directed toward closure devices; more specifically, embodiments are directed toward closure devices for closing an opening in a body vessel.

### BACKGROUND

Arteriotomy closure after diagnostic and/or interventional catheterization procedures has been addressed by a number of devices in addition to manual compression.

For a diagnostic and/or interventional catheterization procedure, such as a coronary procedure, a small gauge needle may be introduced through a patient's skin to a target blood vessel, such as the femoral artery in the region of the patient's groin. The needle forms a puncture, i.e. an arteriotomy, through the blood vessel wall. A guide wire may then be introduced through the needle, and the needle withdrawn over the guide wire. An introducer-sheath may be next introduced over the guide wire, and the sheath may be left in place to provide access during the procedure. Examples of procedures include diagnostic procedures such as angiography, ultrasonic imaging, and the like, and interventional procedures, such as angioplasty, atherectomy, stent placement, cardiac valve procedures, laser ablation, graft placement, and the like.

After the procedure is completed, the catheters, guide wire, and introducer-sheath are removed, and it is necessary to close the arteriotomy to provide hemostasis (i.e., stop blood loss) and allow healing.

### SUMMARY

One or more embodiments of the present disclosure include a closure device for closing an opening in a body vessel. The closure device may include a plug, an intravascular anchor, a suture that couples the plug to the intravascular anchor and a cinching element that provides a compressive axial force to the plug, wherein the plug and the cinching element are positioned over a length of the suture and the suture is secured to the intravascular anchor with a fastening portion embedded in the intravascular anchor.

One or more embodiments of the present disclosure include a system for closing an opening in a body vessel. The system may include a sheath, the closure device having the plug, the intravascular anchor, the suture, the cinching element, and a push member.

One or more embodiments of the present disclosure include a method of making the closure device for closing an opening in a body vessel. The method may include embedding a fastening portion of a suture in an intravascular anchor to secure the suture to the intravascular anchor and positioning a plug and a cinching element over a length of the suture to couple the plug to the intravascular anchor.

The above summary of the present disclosure is not intended to describe each disclosed embodiment or every implementation of the present disclosure. The description that follows more particularly exemplifies illustrative embodiments. In several places throughout the application, guidance is provided through examples, which examples can be used in various combinations. In each instance, the recited list serves only as a representative group and should not be interpreted as an exclusive list.

### BRIEF DESCRIPTION OF THE FIGURES

FIG. 1 illustrates a closure device according to an embodiment of the present disclosure.

FIG. 2 illustrates an embodiment including a fastening portion having a first cross-sectional area that is larger than a second cross-sectional area of a portion of the suture that is not embedded in the intravascular anchor.

FIG. 3 illustrates a fastening portion of the suture that includes a knot according to an embodiment of the present disclosure.

FIG. 4 illustrates a suture with a fastening portion having a first leg, a second leg, and an attachment member according to an embodiment of the present disclosure.

FIG. 5 illustrates a suture with a fastening portion having a first leg, a second leg, and an attachment member according to an embodiment of the present disclosure.

FIG. 6 illustrates a fastening portion of the suture and an additional fastening portion of the suture embedded in the intravascular anchor according to an embodiment of the present disclosure.

FIG. 7 illustrates an embodiment including a second suture.

FIG. 8 illustrates a system for closing an opening in a body vessel according to an embodiment of the present disclosure.

FIG. 9 illustrates the system for closing an opening in a body vessel disposed within an introducer sheath according to an embodiment of the present disclosure.

### DETAILED DESCRIPTION

Embodiments of the present disclosure provide a closure device for closing an opening in a body vessel. The closure device may include a plug, an intravascular anchor, a suture and a cinching element. The suture couples the plug to the intravascular anchor. The cinching element provides a compressive axial force to the plug. The plug and the cinching element are positioned over a length of the suture and the suture is secured to the intravascular anchor with a fastening portion embedded in the intravascular anchor.

Anchor-plug-cinch devices have been employed for arteriotomy closure procedures, e.g., closing an opening in a body vessel. However, there may be complications, such as bleeding or embolism, if the anchor-plug-cinch device does not seat properly against the arteriotomy or if there is a premature fracture, degradation, or release of a portion of the anchor-plug-cinch device. Some previous anchor-plug-cinch devices can be bulky, stiff, or unreliable in order to accommodate proper seating and help avoid premature fracture, degradation, or release of those anchor-plug-cinch devices. Additionally, some anchor-plug-cinch devices may be prone to failure of the anchor as the plug is being cinched. For some anchor-plug-cinch devices this proneness may be at least partially due to a relative motion, e.g. “sawing”, between the anchor and the suture.

The present disclosure describes embodiments of a closure device that may provide secure attachment of the plug to the intravascular anchor, which helps to provide for proper seating against an opening in a body vessel while minimizing the potential for premature fracture, sawing, degradation, and/or release of a portion of the closure devices, as disclosed herein. The present disclosure describes embodiments of a closure device that may improve an opposed force applied via a suture. While adding improved securing properties to the closure devices, embodiments of the present disclosure may also be advantageous, relative to some anchor-plug-cinch devices, by providing a simplified fabrication.

As used herein, “a,” “an,” “the,” “at least one,” and “one or more” are used interchangeably. The term “and/or” means one, one or more, or all of the listed items. The recitations of numerical ranges by endpoints include all numbers subsumed within that range (e.g., 1 to 5 includes 1, 1.5, 2, 2.75, 3, 3.80, 4, 5, etc.).

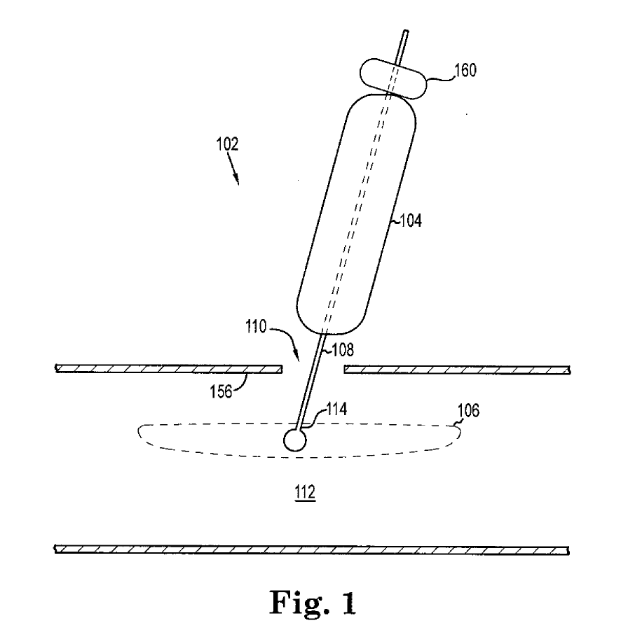

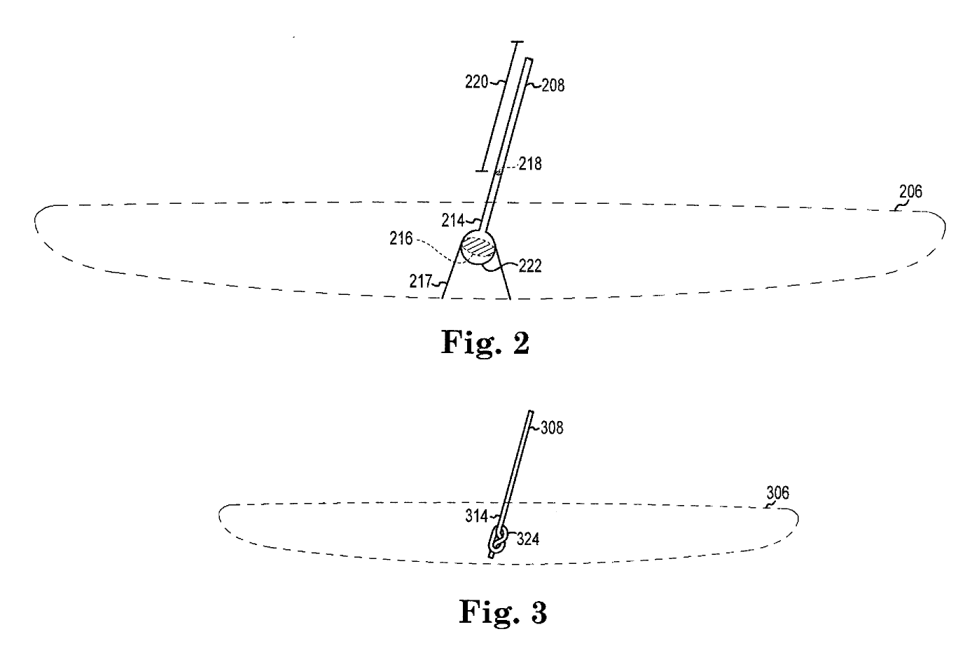

The figures herein follow a numbering convention in which the first digit or digits correspond to the drawing figure number and the remaining digits identify an element in the drawing. Similar elements between different figures may be identified by the use of similar digits. For example, 106 may reference element “06” in FIG. 1, and a similar element may be referenced as 206 in FIG. 2. It is emphasized that the purpose of the figures is to illustrate and the figures are not intended to be limiting in any way. The figures herein may not be to scale and relationships of elements in the figures may be exaggerated. The figures are employed to illustrate conceptual structures and methods herein described.

FIG. 1 illustrates a closure device 102 including a plug 104, an intravascular anchor 106, a suture 108, and a cinching element 160. As FIG. 1 illustrates, the suture 108 couples the plug 104 to the intravascular anchor 106. In a deployed state of the closure device 102, a portion of the suture 108 may pass through an opening 110 in a body vessel 112.

Suture, as used herein, refers to a filament, fibril, or threadlike fastening component. The suture may be monofilament, multifilament, or combinations thereof. The suture may be twisted, braided, or combinations thereof. For one or more embodiments, the suture may be resorbable. Resorbable, as used herein, refers to being degradable within a body. Body vessel, as used herein, refers to a blood vessel or other structure which must be separated from or maintain separation between other tissues and spaces, including, but not limited to, arteries, veins, heart chambers, lymph vessels, ureters, ducts, cerebrospinal fluid spaces, sinuses, respiratory tract, gastrointestinal tract, sheaths, fascia, hollow organs, body walls, portions thereof, combinations thereof, and the like.

For one or more embodiments, the suture 108 engages the intravascular anchor 106 such that the suture 108 is secured to the intravascular anchor 106 with a fastening portion 114 embedded in the intravascular anchor 106. The fastening portion 114 embedded in the intravascular anchor 106 helps provide for proper seating of the closure device 102 against body vessel wall 156 at an opening 110 in the body vessel 112 while minimizing the potential for premature fracture, sawing, degradation, and/or release of a portion of the closure device 102 by increasing the interface area between the suture 108 and the intravascular anchor 106, relative to some anchor-plug-cinch devices, that are subject to force when the closure device is being deployed and/or in the deployed state.

For one or more embodiments, the closure device 102 may include a cinching element 160. The cinching element may include an aperture such that suture 108 may extend from the intravascular anchor 106 through the plug 104 and through the aperture of cinching element 160. For one or more embodiments, the cinching element 160 can be moved longitudinally along the suture 108 to allow for a compressive axial force to be applied to the plug 104. For example, the cinching element 160 can be advanced along the suture 108 towards the anchor 106, where it comes into contact with the plug 102 to apply the compressive axial force.

The cinching element 160 may hold the plug 104 in a deformed state. For example, a friction fit between the cinching element 160 and the suture 108 may be employed. For one or more embodiments, the suture 108 and the cinching element 160 may include a one-way mechanism. The one-way mechanism can help prevent the cinching element 160 from reversibly moving along the suture 108 once the cinching element 160 has passed the one-way mechanism. One-way mechanisms can include, but are not limited to, a ratchet system, one or more barbs, and combinations thereof.

FIG. 2 illustrates an embodiment including a fastening portion 214 having a first cross-sectional area 216 that is larger than a second cross-sectional area 218 of a portion of the suture 208 that is not embedded in the intravascular anchor 206, where the first cross-sectional area 216 and the second cross-sectional area 218 are perpendicular to a longitudinal axis 220 of the suture 208. For some embodiments, as illustrated in FIG. 2, the fastening portion 214 may include a fastening component 222 that includes the first cross-sectional area 216.

For one or more embodiments, a distal end of the fastening portion 214 is flush with a bottom surface of the intravascular anchor 206. Positioning the distal end of the fastening portion 214 flush with the bottom surface of the intravascular anchor 206 can help provide resistance to suture pullout failure when the closure device is subjected to a tensile load.

The fastening component 222 is illustrated as a sphere; however the fastening component may include other shapes. For example, the fastening component 222 may include one or more polyhedron, sphere, cylinder, cone, irregular shape, and combinations thereof. For some embodiments, the fastening component 222 may be formed from the fastening portion 214, while in some other embodiments the fastening component 222 may be a snap fit device or a pressure fit device adapted to couple the fastening component 222 to the fastening portion 214. The fastening portion 214 can include one or more bends or loops. For one or more embodiments, the fastening component 222 may include one or more indentations, e.g. one or more pores, cavities, and/or grooves. For some applications, the one or more indentations may help provide an increased surface area of the fastening component 222 that is embedded and in contact with the intravascular anchor 206.

For one or more embodiments, the first cross-sectional area 216 spans a larger area than the second cross-sectional area 218 of a portion of the suture 208 that is not embedded in the intravascular anchor 206; for example the first cross-sectional area 216 can be from five percent to five hundred percent, or from twenty percent to two hundred percent larger than the second cross-sectional area 218 of a portion of the suture 208 that is not embedded in the intravascular anchor 206.

FIG. 3 illustrates an embodiment including a suture 308 having uniform cross-sectional areas that are perpendicular to a longitudinal axis of the suture 308 throughout the length of the suture 308. As illustrated in FIG. 3, the fastening portion 314 of suture 308 includes a knot 324. Knot, as used herein, refers to an interlacing, a twining, a looping, or combinations thereof of the suture. Because the suture 308 has uniform cross-sectional areas that are perpendicular to a longitudinal axis of the suture 308 throughout the length of the suture 308, knot 324 may be employed to increase a surface area of the suture 308 that is embedded and in contact with the intravascular anchor 308.

It is contemplated that a type of knot, which increase a surface area of the suture that is embedded and in contact with the intravascular anchor, is suitable for the disclosed embodiments. Types of knots include, but are not limited to, bend knots, binding knots, coil knots, loop knots, and combinations thereof. Additionally, for one or more embodiments, the fastening portion of the suture may include a plurality of knots. For example, the fastening portion of the suture may include two, three, four, or even a greater number of knots. For embodiments including the plurality of knots, one or more types of knots may be employed.

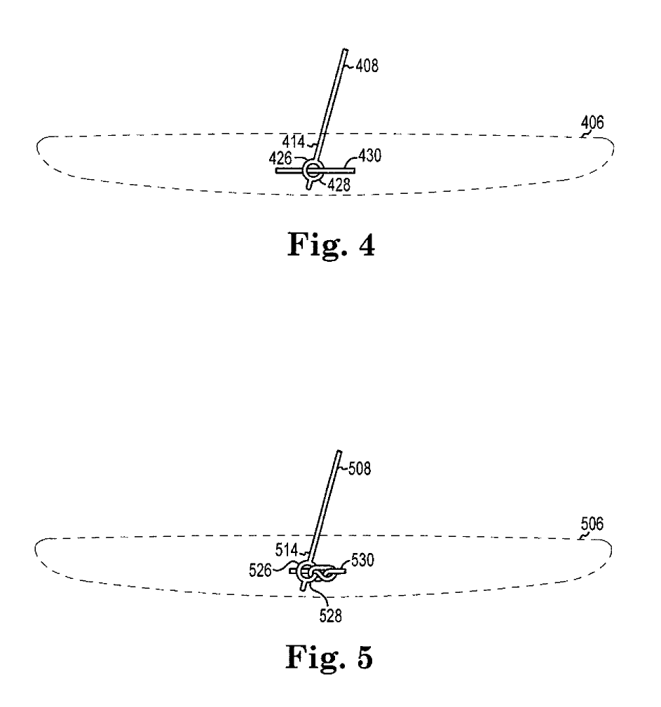

FIG. 4 illustrates an embodiment including a suture 408 with the fastening portion 414 having a first leg 426 and a second leg 428. The first leg 426 and the second leg 428 may diverge from the fastening portion 414. As illustrated in FIG. 4, the first leg 426 and the second leg 428 are separated by a portion of the intravascular anchor 406. However, for some embodiments the first leg 426 and the second leg 428 may diverge from the fastening portion 414 and not be separated by a portion of the intravascular anchor 406. For example, the first leg 426 and the second leg 428 may diverge from the fastening portion 414 and still contact one another. For one or more embodiments, the first leg 426 and the second leg 428 may reconverge.

Embodiments disclosed herein may include an attachment member 430. As illustrated in FIG. 4, the attachment member 430 may be embedded in the intravascular anchor 406 such that a portion of the attachment member 430 may be located between the first leg 426 and the second leg 428. For example, the attachment member 430 may transversely intersect the fastening portion 414 by being located between a point of divergence and a point of reconvergence of the first leg 426 and the second leg 428. The attachment member 430 is illustrated as a cylinder; however the attachment member may include other shapes. For example, the attachment member may include one or more polyhedron, sphere, cylinder, cone, irregular shapes, bends, loops and combinations thereof. For one or more embodiments, the attachment member may include one or more indentations, e.g. one or more pores, cavities, and/or grooves.

For one or more embodiments, the first leg and the second leg may not reconverge. For example, the suture may be splayed within the intravascular anchor to provide a plurality of splayed legs. The plurality of splayed legs may increase a surface area of the suture that is embedded and in contact with the intravascular anchor.

FIG. 5 illustrates an embodiment where the attachment member 530 encompasses at least one of the first leg 526 and the second leg 528. For example, as illustrated in FIG. 5, the attachment member 530 is knotted about the first leg 526.

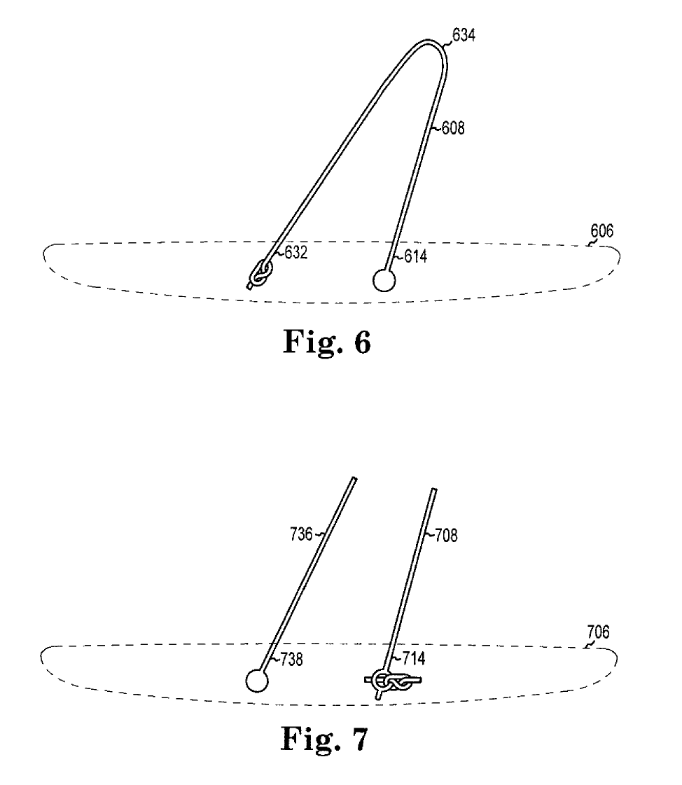

For one or more embodiments, the suture is secured to the intravascular anchor with an additional fastening portion embedded in the intravascular anchor such that the suture forms a loop. FIG. 6 illustrates fastening portion 614 of suture 608 and an additional fastening portion 632 of suture 608 are embedded in the intravascular anchor 606. Because the fastening portion 614 and the additional fastening portion 632 are embedded in the intravascular anchor 606, the suture forms a loop 634, e.g., a closed loop.

For one or more embodiments, the additional fastening portion may have a first additional fastening portion cross-sectional area that is larger than a second cross-sectional area of a portion of the suture that is not embedded in the intravascular anchor. For such embodiments, the first additional fastening portion cross-sectional area and the second cross-sectional area are perpendicular to a longitudinal axis of the suture. For one or more embodiments, the additional fastening portion may include an additional fastening component that includes the first additional fastening portion cross-sectional area. The additional fastening component is similar to, e.g. sharing the properties of, the fastening component, as discussed herein.

Like the first cross-sectional area, the first additional fastening portion cross-sectional area spans a larger area than the second cross-sectional area of the portion of the suture that is not embedded in the intravascular anchor; for example the first additional fastening portion cross-sectional area can be from five percent to five hundred percent, or from twenty percent to two hundred percent larger than the second cross-sectional area of a portion of the suture that is not embedded in the intravascular anchor.

For one or more embodiments, the additional fastening portion may include a knot, as discussed herein. Additionally, for one or more embodiments, the additional fastening portion of the suture may include a plurality of knots. For example, the additional fastening portion of the suture may include two, three, four, or even a greater number of knots. For embodiments including the plurality of knots, one or more types of knots may be employed.

For one or more embodiments, the additional fastening portion may include a first additional fastening portion leg and a second additional fastening portion leg. These legs are similar to, e.g. sharing the properties of, the first leg and the second leg, as discussed herein.

One or more embodiments including the additional fastening portion may include an additional attachment member. The additional attachment member is similar to, e.g. sharing the properties of, the attachment member, as discussed herein.

FIG. 7 illustrates an embodiment including a second suture 736. The second suture 736 is similar to, e.g. sharing the properties of, the suture 708, as discussed herein.

The second suture 736 may include a second suture fastening portion 738. The second fastening portion 738 is similar to, e.g. sharing the properties of, the fastening portion 714. One or more embodiments may include a second fastening component. The second fastening component is similar to, e.g. sharing the properties of, the fastening component, as discussed herein. One or more embodiments may include a second attachment member. The second attachment member is similar to, e.g. sharing the properties of, the attachment member, as discussed herein.

For one or more embodiments, the suture may degrade within a body in a period of time of 3 days to 180 days. For some applications the suture may degrade within the body in a period of time of 60 days to 90 days. For one or more embodiments, the suture may include a suture material selected from the group consisting of esters, sugars, biological materials, and combinations thereof.

For one or more embodiments, the suture may include an ester, e.g. a polyester. Examples of esters include, but are not limited to, polyglycolic acid, polylactic acid, poly(lactic-co-glycolic acid), polycaprolactone, polydioxanone, and combinations thereof.

For one or more embodiments, the suture may include a sugar. Sugar, as used herein refers to carbohydrates including monosaccharides, disaccharides, oligosaccharides, and polysaccharides having, for example, four (tetrose), five (pentose), six (hexose), seven (heptose), or more carbon atoms, and combinations thereof. Examples of monosaccharides include, but are not limited to, allose, altrose, glucose, mannose, gulose, idose, galactose, talose, ribose, arabinose, xylose, lyxose, erythrose, threose, glyceraldehyde, and combinations thereof. Examples of disaccharides include, but are not limited to, cellobiose, maltose, lactose, gentiobiose, sucrose, and combinations thereof. Examples of oligosaccharides and/or polysaccharides include, but are not limited to, cellulose, starch, amylase, amylopectin, glycogen, and combinations thereof.

For one or more embodiments, the suture may include a biological material. Examples of biological materials include, but are not limited to, surgical gut, e.g. catgut, silk, and combinations thereof. For one or more embodiments, the biological material may be treated with a chromium salt solution to provide a chromatic suture material. For one or more embodiments, the suture may include protein material, derivatives, or synthetic analogs, such as collagen or modified collagen.

The suture may have a generally circular cross section. For one or more embodiments, the suture may have a diameter of 0.18 millimeters to 0.70 millimeters; for example the suture may have a diameter of 0.25 millimeters to 0.50 millimeters, or 0.30 millimeters to 0.40 millimeters. The suture may have a diameter, as discussed herein, at one or more portions of the suture. For one or more embodiments, the suture may have a uniform diameter. For one or more embodiments, the suture may have a plurality of diameters corresponding to different values, as discussed herein. The suture may have a generally non-circular, e.g., rectangular, triangular, elliptical, etc., cross section. For embodiments having a generally non-circular cross section the cross-sectional area is as described for embodiments having the generally circular cross section.

For one or more embodiments, the intravascular anchor, as disclosed herein, may be resorbable, e.g. the intravascular anchor may degrade within a body in a period of time of 3 days to 180 days. For some applications the intravascular anchor may degrade within the body in a period of time of 45 days to 90 days.

For one or more embodiments, the intravascular anchor may include an ester, as discussed herein. For one or more embodiments, the intravascular anchor may include a sugar, as discussed herein. For one or more embodiments, the intravascular anchor may include a biological material, as discussed herein. For example, the intravascular anchor may include an intravascular anchor material selected from the group consisting of esters, sugars, and combinations thereof.

For one of more embodiments, the intravascular anchor consists of a malleable biodegradable polyurethane elastomer (condensation product from mixed dihydric and trihydric alcohols and di-isocyanates such as cyclohexane di-isocyanate). However, it may be desirable that the intravascular anchor is rigid, e.g. not deformable, during deployment and/or when tension is applied to help ensure that the intravascular anchor is not inadvertently removed from an opening in a body vessel.

For various applications, the intravascular anchor may have differing shapes. For one or more embodiments, the intravascular anchor may include one or more polyhedron, sphere, cylinder, cone, and combinations thereof. For some applications, the intravascular anchor may have a first surface configured to appose the body vessel wall. For example, the first surface may be convex in relation to the intravascular anchor such that the convex first surface conforms to a concave surface of the body vessel. For some applications, the intravascular anchor may have a second surface configured to help minimize flow disturbances and/or flow separation within the body vessel. For example, the second surface may be canted, e.g. where a first end and a second end of the intravascular anchor have a thickness that is less than a thickness at the center of the intravascular anchor.

For one or more embodiments, the intravascular anchor may include an indentation 217, e.g. a trough or a dimple. The indentation may be located, for example on the second surface, as discussed herein. For one or more embodiments including the indentation, a portion of the suture, a portion of the knot, or a portion of the attachment member may be at least partially located within the indentation. The portion of the suture, portion of the knot, or portion of the attachment member at least partially located within the indentation may oppose a force applied via a suture.

The indentation may be formed during manufacture of the intravascular anchor. For example, a mold employed to manufacture the intravascular anchor may include one or more surfaces configured to define the indentation. The indentation may be totaled by removing a portion of the intravascular anchor. For example, a portion of the intravascular anchor may be removed, e.g. cut from, drilled from, or another removal process, from the intravascular anchor to provide the indentation. For one or more embodiments, the intravascular anchor may include a dome. The dome of the intravascular anchor can help provide resistance to suture pullout failure when the closure device is subjected to a tensile load.

For various applications the intravascular anchor may have differing dimensions. For one or more embodiments, the intravascular anchor may have a length of 2 millimeter to 25 millimeters; for example the intravascular anchor may have a length of 5 millimeters to 18 millimeters, or 8 millimeters to 13 millimeters. For one or more embodiments, the intravascular anchor may have a width of 1 millimeter to 8 millimeters; for example the intravascular anchor may have a width of 1 millimeter to 5 millimeters, or 1.5 millimeters to 2.5 millimeters. For one or more embodiments, the intravascular anchor may have a thickness of 0.25 millimeters to 5 millimeters; for example the intravascular anchor may have a thickness of 0.5 millimeters to 3 millimeters, or 0.75 millimeter to 2 millimeters.

For one or more embodiments, the plug includes a plug material. For some applications, the plug material may include a clot promoting material. Examples of the plug material include, but are not limited to a collagen foam, a starch powder, and combinations thereof. For one or more embodiments, the plug may include from 10 weight percent collagen foam to 50 weight percent collagen foam and from 90 weight percent starch powder to 50 weight percent starch powder; for example the plug may include 20 weight percent collagen foam and 80 weight percent starch powder, 30 weight percent collagen foam and 70 weight percent starch powder, or 40 weight percent collagen foam and 60 weight percent starch powder.

For one or more embodiments, the plug, as disclosed herein, may be resorbable, e.g. the plug may degrade within a body in a period of time of 3 days to 180 days. For some applications the plug may degrade within the body in a period of time of 30 days to 70 days. For one or more embodiments including a combination of plug materials, e.g. an embodiment that includes collagen foam and starch powder, differing portions of the plug may degrade within the body at different rates and/or in a different period of time. For example, a first portion of the plug may degrade within the body in a period of time of 2 days to 3 days and a second portion of the plug may degrade within the body in a period of time of 45 days to 75 days.

The plug may have an undeformed state, e.g. prior to the plug engaging the body vessel and/or a portion of the intravascular anchor. The plug may have a deformed state, e.g. when engaging the body vessel and/or a portion of the intravascular anchor.

For various applications, the plug in the undeformed state may have differing shapes. For one or more embodiments, plug in the undeformed state may include one or more polyhedron, sphere, cylinder, cone, and combinations thereof. For some preferred embodiments, the plug in the undeformed state may have a cylindrical shape.

For various applications the plug in the undeformed state may have differing dimensions. For one or more embodiments, plug in the undeformed state may have a length of 0.5 centimeters to 5 centimeters; for example the plug in the undeformed state may have a length of 1 centimeter to 4 centimeters, or 1.5 centimeters to 3 centimeters. For some embodiments, the plug in the undeformed state may have a length of 1.8 centimeters to 2.0 centimeters. The plug may have a generally circular cross section. For one or more embodiments, plug in the undeformed state may have a diameter of 0.04 centimeters to 0.30 centimeters; for example the plug in the undeformed state may have a diameter of 0.10 centimeters to 0.24 centimeters, or 0.14 centimeters to 0.20 centimeters. The plug may have a generally rectangular cross section. For embodiments having a generally rectangular cross section the cross-sectional area is as described for embodiments having a generally circular cross section.

The fastening component, as disclosed herein, may be resorbable, e.g. the fastening component may degrade within a body in a period of time of 3 days to 180 days. For some applications the fastening component may degrade within the body in a period of time of 45 days to 90 days.

For one or more embodiments, the fastening component may include an ester, as discussed herein. For one or more embodiments, the fastening component may include a sugar, as discussed herein. For one or more embodiments, the fastening component may include a biological material, as discussed herein.

The attachment member, as disclosed herein, may be resorbable, e.g. the attachment member may degrade within a body in a period of time of 3 days to 180 days. For some applications the attachment member may degrade within the body in a period of time of 45 days to 90 days.

For one or more embodiments, the attachment member may include an ester, as discussed herein. For one or more embodiments, the attachment member may include a sugar, as discussed herein. For one or more embodiments, the attachment member may include a biological material, as discussed herein.

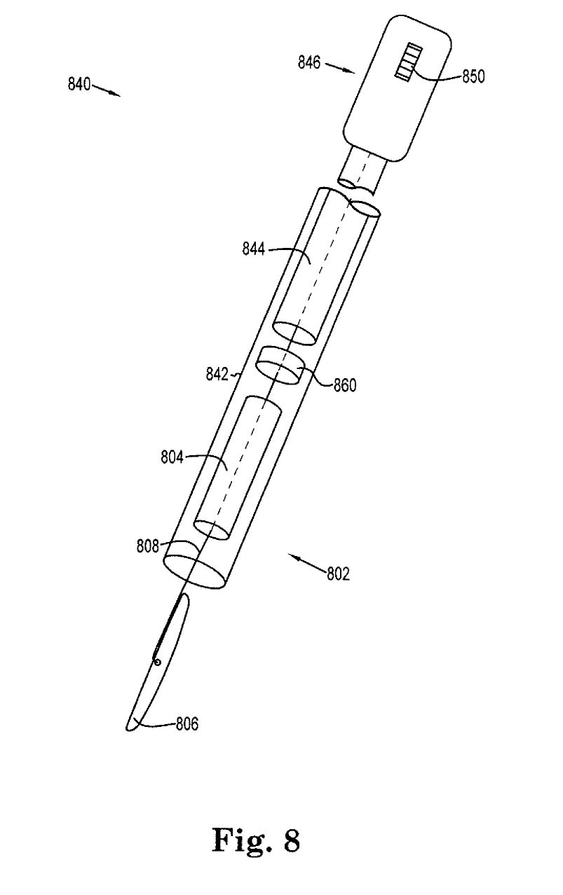

Embodiments of the present disclosure provide a system for closing an opening in a body vessel. FIG. 8 illustrates a system 840 for closing an opening in a body vessel. The system includes a device sheath 842, a closure device 802, and a push member 844.

Push member 844 is disposed in the sheath 842 and configured to deploy the closure device 802. Push member 844 may be located, for example, adjacent the closure device 802. While the push member is illustrated as a generally cylindrical elongate member, the push member may include one or more polyhedron, sphere, cylinder, cone, and combinations thereof.

Closure device 802 may be releasably housed in the device sheath 842. Closure device 802 may include a plug 804, an intravascular anchor 806, a cinching element 860, and a suture 808 that couples the plug 804 to the intravascular anchor 806, where the plug 804 is positioned over a length of the suture 808 and the suture 808 is secured to the intravascular anchor 806 with a fastening portion embedded in the intravascular anchor 806. Cinching element 860 may retain the plug 804 in a deployed state and/or resist movement along the suture 808. Examples of cinching element 860 include, but are not limited to, a disc including an aperture, a sliding knot, another bonding mechanism, another latching mechanism, and combinations thereof. The intravascular anchor 806 may include a furrow and/or channel along a portion of the intravascular anchor. The furrow and/or channel may be located along a central portion of the intravascular anchor. The intravascular anchor including the furrow and/or channel may be foldable within the sheath.

For one or more embodiments, the system 840 may include a handle 846. Handle 846 may include one or more control members, such as an actuation member 850. Examples of actuation member 850 include, but are not limited to, sliders, buttons, levers, and switches. The one or more control members may be coupled the intravascular anchor 806 and may help control positioning of the intravascular anchor 806. Handle 846 may also include a number of different and/or alternative structural features. The actuation member 850 may function, among other things, to retract the sheath 842, to deploy the plug 804, to apply a tension force to the suture 808, and/or to cut the suture 808.

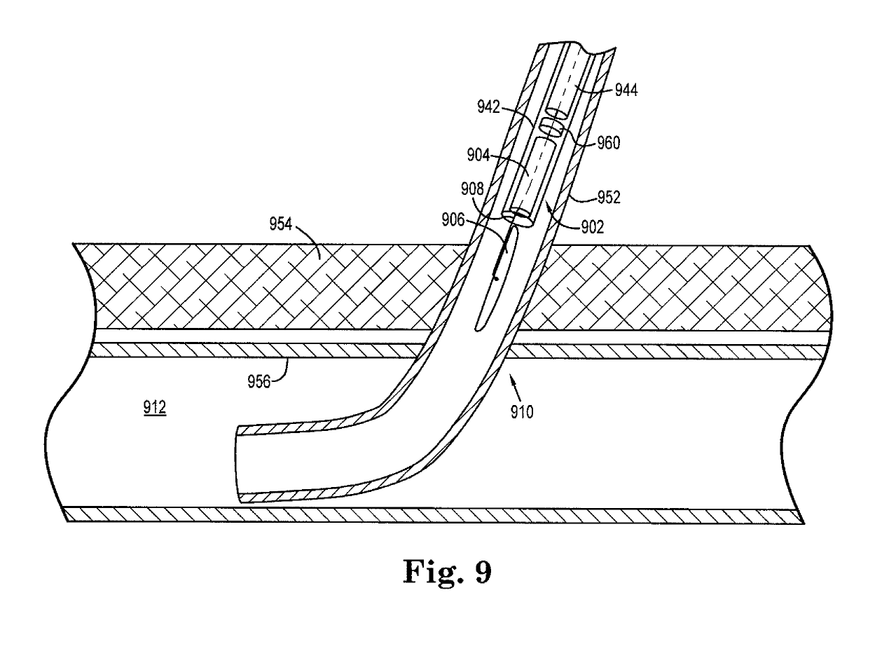

FIG. 9 illustrates the system disposed within an introducer sheath 952. As illustrated in FIG. 9, the introducer sheath 952 extends through the skin and tissue 954 and into the body vessel 912, e.g. the femoral artery. Deployment of the system may include the use of an obturator and/or dilator.

For some applications, the system including device sheath 942, anchor 906, plug 904, cinching element 960 may be advanced through the introducer sheath 952 to a position where the closure device 902 may be advanced out from the introducer sheath 952 and into the body vessel 912.

After and/or while being advanced out from the introducer sheath 952, the intravascular anchor 906 may be configured to shift and/or tilt to prepare for engagement with body vessel wall 956. The shifting and/or tilting may be accomplished in a number of different ways. In one embodiment, fluid expansion of the plug 904 may provide energy to move the intravascular anchor 906 towards a desired position. In another embodiment, suture 908 may be configured or otherwise be arranged in conjunction with the intravascular anchor 906 so that the suture 908 may be manipulated to cause the intravascular anchor 906 to shift and/or tilt. For some applications, the suture 908 may be wrapped and/or wound around one or more portions of the intravascular anchor 906.

For some applications, device sheath 942, together with intravascular anchor 906, plug 904, cinching element 960, and suture 908 are retracted, e.g. moved proximally, until intravascular anchor 906 is seated against the end of introducer sheath 952. Introducer sheath 952, device sheath 942, intravascular anchor 906, plug 904, and suture 908 are then retracted, e.g. moved proximally, until anchor 906 is seated against the body vessel wall 956. Introducer sheath 952 is then retracted, e.g. moved proximally, providing a gap for deployment of plug 904. In some embodiments, device sheath 942 is retracted, e.g. moved proximally, exposing plug 904 for deployment. In other embodiments, device sheath 942 is configured to deform during plug deployment, allowing the plug 904 to move outward for deployment while displacing a portion of device sheath 942, e.g. moving it out of the way (this may be referred to as a shoehorn sheath).

As discussed herein, a tension force may be applied to the suture 908. The tension force may be applied prior to, concurrently with, and/or after the push force is applied to the plug 904. Application of the tension force may pull together and/or secure the intravascular anchor 906 with the plug 904. For one or more embodiments, during application of the tension force the suture 908 does not move relative to the intravascular anchor 906. Once desirably situated, e.g. the intravascular anchor 906 engaging the body vessel wall and the deformed plug 904 engaging the body vessel and/or a portion of the intravascular anchor 906, the device sheath 942 and/or the introducer sheath 952 may be retracted to leave the closure device 902 closing the opening in the body vessel. Excess suture, e.g. a portion of suture extending from the desirably situated closure device, may be removed.

One or more embodiments, as disclosed herein, may include a bioactive coating. Bioactive, as used herein, refers to having a capacity to interact with living tissue, such as vascular tissue. One example of interacting with living tissue is bonding to the living tissue. This bonding may help provide adhesion properties that may be desirable for some applications.

Vascular tissue, e.g. body vessels, may contain approximately two to five weight percent of glycosaminoglycans based upon the total non-aqueous material of the vascular tissue. For an injured vasculature, e.g. where a vascular basement membrane is exposed, the vascular basement membrane is composed of 50% collagen IV. Glycosaminoglycans, which may also be referred to as mucopolysaccharides, are unbranched polysaccharides including a repeating disaccharide unit. The repeating disaccharide unit includes a hexose, e.g. a six-carbon sugar, or a hexuronic acid, linked to a hexosamine, e.g. a six-carbon sugar containing nitrogen.

Peptides that include a grouping of positive charge can bond to the glycosaminoglycans of the vascular tissue. For one or more embodiments, the peptide and/or a peptide sequence may be specifically selected to bind to collagen IV. Examples of peptides having a grouping of positive charge include, but are not limited to, peptides containing a linear trimer of a consensus sequence. An example of a peptide containing a linear trimer of a consensus sequence includes, but is not limited to, (Alanine-Arginine-Arginine-Arginine-Alanine-Alanine-Arginine-Alanine)₃, which may also be written (ARRRAARA)₃. This 24 mer peptide has a mass of 2.7 kilodalton (kDa).

For one or more embodiments, the intravascular anchor includes the bioactive coating. Coating, as used herein, refers to a layer of substance on a surface and further includes surfaces exposing a desired functional group.

For some applications, the intravascular anchor is coated with the bioactive coating. For example, the peptide can be combined with other materials, e.g. the intravascular anchor material, to form a combination, such as a homogenous mixture, that will constitute the intravascular anchor. The intravascular anchor, formed from the homogenous mixture, will have surfaces exposing a desired functional group, e.g. desired functional groups of peptides, and thus be essentially completely coated with the bioactive coating.

For some applications, the intravascular anchor is partially coated with the bioactive coating. For example, the peptide can be applied to desirable locations on the intravascular anchor, such as surfaces of the intravascular anchor that are configured to contact the body vessel and/or the plug.

For some applications, the surface of the intravascular anchor may be chemically activated by the introduction of specific functional groups at the surface that allows for one end of the peptide sequence to be chemically coupled to the surface. For example, the peptide may be covalently bonded to the surface of the intravascular anchor that is designed to contact with the body vessel.

For one or more embodiments, the plug includes the bioactive coating. For some applications, the plug is essentially completely coated with the bioactive coating. For example, the peptide can be combined with other materials to form a combination, e.g. a homogenous mixture that will constitute the plug. The plug, formed from the homogenous mixture, will have surfaces exposing a desired functional group, e.g. desired functional groups of peptides, and thus be essentially completely coated with the bioactive coating.

For some applications, the plug can be partially coated with the bioactive coating. For example, a peptide, polyethylene glycol and/or polyethylene glycol peptide coating can be applied to desirable locations on the plug, such as surfaces of the intravascular anchor that are configured to contact the body vessel and/or the intravascular anchor. For one or more embodiments, the coating may be selected from the group consisting of polyethylene glycol, a polyethylene glycol peptide, a peptide with a consensus sequence (Alanine-Arginine-Arginine-Arginine-Alanine-Alanine-Arginine-Alanine)3, and combinations thereof.

Embodiments of the present disclosure provide methods of making a closure device for closing an opening in a body vessel. A method of making the closure device may include embedding a fastening portion of a suture, as discussed herein, in an intravascular anchor, as discussed herein, to secure the suture to the intravascular anchor. The method may include positioning a plug, as discussed herein, over a length of the suture to couple the plug to the intravascular anchor.

Embedding the fastening portion may include molding the intravascular anchor. Molding the intravascular anchor may include casting, injection molding, another molding process, and combinations thereof. A mold having surfaces configured to define the intravascular anchor may be employed. The fastening portion of the suture may be located within the mold and tensioned, e.g. at both ends of the suture. For some applications the suture may pass entirely through the mold cavity. The intravascular anchor material, as discussed herein, may then be provided to the mold including the tensioned suture.

The intravascular anchor material and the tensioned suture may be maintained within the mold for a period of time. The period of time may differ for various applications and may depend, at least in part, upon which particular intravascular anchor materials are employed. The section of the suture exiting the bottom of the intravascular anchor can be trimmed, e.g. made flush with the bottom surface of the intravascular anchor. This trimmed suture section and/or area may be smoothed over, e.g. with a dispersion or melt of the material used to form the intravascular anchor.

Additionally, for one or more embodiments, maintaining the intravascular anchor material and the tensioned suture may include an application and/or a removal of heat. For example, for some applications while the intravascular anchor material and the tensioned suture are maintained within the mold the mold may be heated. For various applications differing values of heat may be applied, depending, at least in part, upon which particular intravascular anchor materials are employed. For some applications while the intravascular anchor material and the tensioned suture are maintained within the mold the mold may be cooled. For various applications differing values of heat removal may be applied, depending, at least in part, upon which particular intravascular anchor materials are employed.

Furthermore, for one or more embodiments, maintaining the intravascular anchor material and the tensioned suture may include an application of pressure. For example, for some applications while the intravascular anchor material and the tensioned suture are maintained within the mold the mold may be pressed. For various applications differing values of pressure may be applied and may depend, at least in part, upon which particular intravascular anchor materials are employed.

The method may include applying a coating, as discussed herein, to the intravascular anchor and/or the plug. For one or more embodiments, applying the coating may include combining the peptide with the intravascular anchor material. The peptide and the intravascular anchor material may be combined to form a homogenous mixture. For some applications the homogenous mixture may be provided to the mold including the tensioned suture for molding, as discussed herein.

For one or more embodiments, applying the coating may include applying the peptide to a desirable location on the intravascular anchor, such as a surface of the intravascular anchor that is configured to contact the body vessel and/or the plug. For various applications, the coating may be applied in differing processes. Examples of processes for applying the coating include, but are not limited to, dipping, spraying, brushing, chemically binding one end of the peptide via covalent bonds at the surface of the intravascular anchor, and combinations thereof.

It is to be understood that the above description has been made in an illustrative fashion, and not a restrictive one.

What is claimed:

1. A closure device for closing an opening in a body vessel comprising:
a plug;
an intravascular anchor;
a suture that couples the plug to the intravascular anchor; and
a cinching element that provides a compressive axial force to the plug, wherein the plug and the cinching element are positioned over a length of the suture and the suture is secured to the intravascular anchor with a fastening portion embedded in the intravascular anchor.

2. The closure device of claim 1, wherein the fastening portion has a first cross-sectional area that is larger than a second cross-sectional area of a portion of the suture that is not embedded in the intravascular anchor, wherein the first cross-sectional area and the second cross-sectional area are perpendicular to a longitudinal axis of the suture.

3. The closure device of claim 1, further comprising a bioactive coating on the intravascular anchor or the plug, wherein the bioactive coating can bond to the body vessel.

4. The closure device of claim 3, wherein the intravascular anchor coating includes a peptide.

5. The closure device of claim 4, wherein the peptide includes a consensus sequence (Alanine-Arginine-Arginine-Arginine-Alanine-Alanine-Arginine-Alanine)₃.

6. The closure device of claim 1, wherein the fastening portion includes a knot in the suture.

7. The closure device of claim 1, wherein the fastening portion includes a first leg and a second leg, wherein the first leg and the second leg are separated by a portion of the intravascular anchor.

8. The closure device of claim 7, further comprising an attachment member embedded in the intravascular anchor, wherein a portion of the attachment member is located between the first leg and the second leg.

9. The closure device of claim 8, wherein the attachment member encompasses at least one of the first leg and the second leg.

10. The closure device of claim 1, wherein the suture is secured to the intravascular anchor with an additional fastening portion embedded in the intravascular anchor such that the suture forms a loop.

11. The closure device of claim 1, further comprising a second suture that couples the plug to the intravascular anchor, wherein the plug is positioned over a length of the second suture and the second suture is secured to the intravascular anchor with a second fastening portion embedded in the intravascular anchor.

12. The closure device of claim 1, wherein the plug includes a coating selected from the group consisting of polyethylene glycol, a polyethylene glycol peptide, a peptide with a consensus sequence (Alanine-Arginine-Arginine-Arginine-Alanine-Alanine-Arginine-Alanine)3, and combinations thereof.

13. A system for closing an opening in a body vessel comprising:
a device sheath;
a closure device releasably housed in the device sheath, the closure device including
a plug;
an intravascular anchor;
a suture that couples the plug to the intravascular anchor;
a cinching element that provides a compressive axial force to the plug, wherein the plug is positioned over a length of the suture and the suture is secured to the intravascular anchor with a fastening portion embedded in the intravascular anchor; and
a push member disposed in the device sheath, wherein the push member is configured to deploy the closure device.

14. The system of claim 13, wherein the plug includes a biological material; the intravascular anchor includes an intravascular anchor material selected from the group consisting of esters, sugars, and combinations thereof; and the suture includes a suture material selected from the group consisting of esters, sugars, biological materials, and combinations thereof.

15. The system of claim 14, wherein the intravascular anchor includes a combination of the intravascular anchor material and a peptide with a consensus sequence (Alanine-Arginine-Arginine-Arginine-Alanine-Alanine-Arginine-Alanine)₃.

16. The system of claim 13, wherein the intravascular anchor includes a coating having a peptide with a consensus sequence (Alanine-Arginine-Arginine-Arginine-Alanine-Alanine-Arginine-Alanine)₃.

17. The system of claim 13, wherein the plug includes a coating having a peptide with a consensus sequence (Alanine-Arginine-Arginine-Arginine-Alanine-Alanine-Arginine-Alanine)₃.

18. A method of making a closure device for closing an opening in a body vessel, comprising:
embedding a fastening portion of a suture in an intravascular anchor to secure the suture to the intravascular anchor; and
positioning a plug over a length of the suture to couple the plug to the intravascular anchor.

19. The method of claim 16, wherein embedding the fastening portion includes molding the intravascular anchor.

20. The method of claim 17, further comprising applying a coating having a peptide with a consensus sequence (Alanine-Arginine-Arginine-Arginine-Alanine-Alanine-Arginine-Alanine)₃ to the intravascular anchor or the plug.
